(19) United States
(12) Patent Application Publication
(10) Pub. No.: US 2024/0043896 A1
(43) Pub. Date: Feb. 8, 2024

(54) CONTINUOUS HIGH CELL-DENSITY CULTURE WITH DUAL-VESSEL TANGENTIAL FLOW FILTRATION

(71) Applicant: AGC BIOLOGICS, INC., Bothell, WA (US)

(72) Inventors: Alexey Gilman, Bothell, WA (US); John Fann, Bothell, WA (US); Seagan Keefe, Bothell, WA (US)

(21) Appl. No.: 18/031,736

(22) PCT Filed: Oct. 14, 2021
(86) PCT No.: PCT/US2021/054915

§ 371 Date: Apr. 13, 2023

Related U.S. Application Data
(60) Provisional application No. 63/092,327, filed on Oct. 15, 2020.

(51) Int. Cl. C12P 21/00; C12M 1/00; B01D 61/14; B01D 69/02

(52) U.S. Cl. CPC C12P 21/005; C12M 47/10; B01D 61/147; B01D 69/02; B01D 2325/02834; B01D 2315/10

(57) ABSTRACT

The process described herein produces and purifies one or more biomolecules of interest that has practical applications in the field of recombinant protein production in microbial fermentations and mammalian cell culture bioreactors.

### CROSS REFERENCE TO RELATED APPLICATION

This application claims priority to U.S. Provisional Patent Application No. 63/092,327, filed on Oct. 15, 2020, which is incorporated by reference in its entirety.

### FIELD OF THE INVENTION

This invention is directed to processes for producing a biomolecule and has practical applications in the field of recombinant protein production in microbial fermentations and mammalian cell culture bioreactors.

### BACKGROUND OF THE INVENTION

The most common cultivation modes used in biomanufacturing of biomolecules are fed-batch and perfusion (Biotechnol Adv. 2018 July-August; 36(4):1328-1340). One of the most prominent trends in biomanufacturing of mammalian cells is the shift towards continuous bioprocessing using continuous cell culture to enhance product throughput, process speed and minimize costs (Biotechnol Adv. 43 (2020) 107552). An example of a special perfusion process is the continuous perfusion process. The waste products/impurities in the medium are continuously removed from the culture and the displaced medium is replenished. The constant addition medium and elimination of waste products provides the cells with the environment they require to achieve high cell concentrations and with that higher productivity. Thus, it is possible to achieve a state of equilibrium in which cell concentration and productivity are maintained. Product may be continuously harvested by taking out medium, and the cell concentration and viability maintained at high levels via a so-called cell-bleed. However, bottlenecks of continuous perfusion culture remain such as the burden of processing of the high volume of harvest material. Additionally, biomolecule stability may become a problem unless the harvest material is also purified continuously or is stored in conditions that are stable to the biomolecule.

### SUMMARY OF THE INVENTION

The disclosure provides processes for producing a biomolecule. In various embodiments, the disclosure provides a process for producing one or more biomolecules of interest comprising the following steps: (a) culturing cells in a cell culture vessel, where the cell culture vessel is continuously perfused with enriched cell culture medium, and where the cells are cultured under conditions that allow the production of one or more biomolecules of interest. The process produces a cell culture composition comprising the cells, medium and biomolecules. Step (b) comprises passing the cell culture composition of (a) through a first filter, where the first filter retains the cells but allows the passage of medium and biomolecules into a biomolecule accumulation vessel, thereby producing a biomolecule composition. Step (c) comprises concentrating the biomolecules by passing the biomolecule composition of (b) through a second filter, where the second filter retains and returns the biomolecules to the biomolecule accumulation vessel but allows removal of spent medium into a waste collection vessel. Finally, step (d) comprises harvesting the biomolecules from the biomolecule accumulation vessel while continuing steps (a)-(c); thereby purifying one or more biomolecules of interest. In various embodiments, the cells are eukaryotic or prokaryotic cells.

In various embodiments, the first filter is operably attached to the cell culture vessel. In various embodiments, the second filter is operably attached to the biomolecule accumulation vessel. In various embodiments, the pore size of the first filter is 0.2-1 μm. In various embodiments, the pore size of the first filter is 0.5 μm. In various embodiments, the pore size of the second filter is 10-200 kD. In various embodiments, the pore size of the second filter is 50 kD.

In various embodiments, the first filter is an Alternating Tangential Flow (ATF) filter. In various embodiments, the second filter is an Alternating Tangential Flow (ATF) filter. In various embodiments, the first filter is a Tangential Flow Filtration (TFF) filter. In various embodiments, the second filter is a Tangential Flow Filtration (TFF) filter. In various embodiments, the first filter and the second filter are each Alternating Tangential Flow (ATF) filters. In related embodiments, the first filter and the second filter are each Tangential Flow Filtration (TFF) filters. In related embodiments, the first and second filter tangential flow is 0.5-1000 L/min.

In various embodiments, the cell culture in the cell culture vessel is stable for 20-120 days. In related embodiments, the cell culture in the cell culture vessel is maintained for 30-60 days.

In various embodiments, the processes described herein further comprise the biomolecule of interest is concentrated in the biomolecule accumulation vessel. In various embodiments, the pH, temperature, agitation, constant air overlay, dO₂, or CO₂ conditions in the biomolecule accumulation vessel are varied between pH 4-10, 10-40° C., 10-400 revolutions per minute (RPM), 10-100,000 standard cubic centimeters per minute air overlay (sccm), 0-100% dO₂, and/or 0-100% CO₂. In various embodiments, the biomolecule accumulation vessel optionally comprises a buffer exchange claim.

In various embodiments, the harvesting step (d) occurs continuously while performing steps (a), (b) and (c). In related embodiments, the harvesting step (d) occurs every 1-60 days.

In various embodiments, the cell culture vessel is a constant stir tank reactor (CSTR). In various embodiments, the biomolecule accumulation vessel is a constant stir tank reactor (CSTR) or a harvest bag.

In various embodiments, the process produces and purifies one biomolecule of interest. In related embodiments, the process produces and purifies two, three or four biomolecules of interest.

In various embodiments, the biomolecule is a polypeptide, polysaccharide, polypeptide/polysaccharide hybrid, Fc fusion polypeptide, polynucleotide, cytokine, growth factor, hormone, enzyme, vaccine, anticoagulation factor or small molecule. In related embodiments, the polypeptide is an antibody, antibody fragment, antibody fusion peptide or antigen-binding fragments thereof.

In various embodiments, the disclosure provides a process for producing a biomolecule comprising: (a) culturing cells in a cell culture vessel, wherein the cell culture vessel is continuously perfused with enriched cell culture medium, and wherein the cells are cultured for 30-60 days under conditions that allow the production of a biomolecule, thereby producing a cell culture composition comprising the cells, medium and biomolecules; (b) passing the cell culture composition of (a) through a first 0.2 μm hollow fiber filter wherein the first hollow fiber filter retains the cells but allows the passage of biomolecules and medium into a biomolecule accumulation vessel, thereby producing a biomolecule composition; (c) concentrating the biomolecules by passing the biomolecule composition of (b) through a second 50 kD hollow fiber filter, wherein the second hollow fiber filter retains and returns the biomolecules to the biomolecule accumulation vessel but allows removal of spent medium into a waste collection vessel and wherein the pH, temperature, agitation, constant air overlay, dO₂, or CO₂ conditions in the biomolecule accumulation vessel are varied between pH 4-10, 30-40° C., 10-400 RPM, 10-100,000 sccm, 0-100% dO₂, and/or 0-100% CO₂; and (d) harvesting the biomolecules from the biomolecule accumulation vessel while continuing steps (a)-(c); thereby purifying the biomolecule.

### BRIEF DESCRIPTION OF THE DRAWINGS

FIG. 1 shows a schematic of an exemplary dual vessel culture system for performing the process of producing a biomolecule disclosed herein.

FIGS. 2A-2C show viable cell density (VCD) (FIG. 2A), cell viability (FIG. 2B) and cell titer (FIG. 2C) parameters measured during continuous perfusion cell culture.

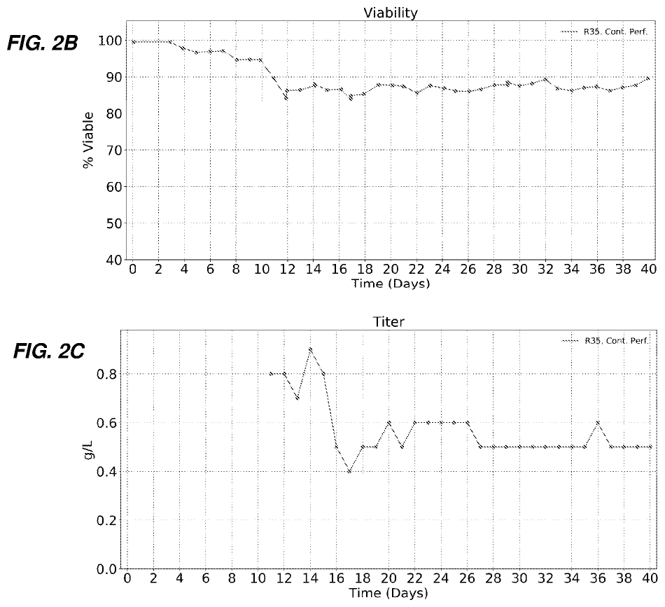

FIG. 3 shows product accumulation (titer) during continuous perfusion cell culture.

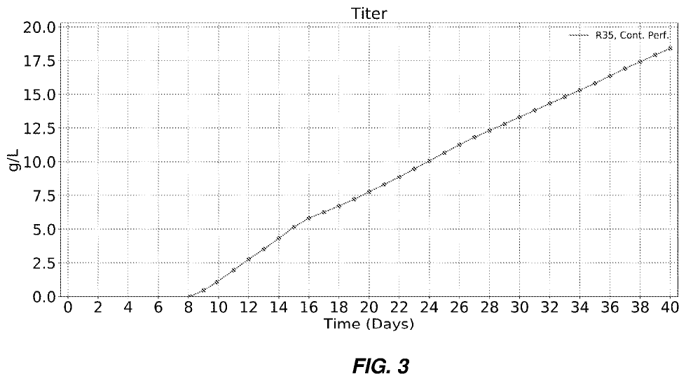

FIGS. 4A-4D show measurement of primary metabolic profile parameters glucose (Gluc) (FIG. 4A), lactate (Lac) (FIG. 4B), Osmolality (Osm) (FIG. 4C), partial pressure of carbon dioxide (PCO₂) (FIG. 4D) levels during continuous perfusion cell culture.

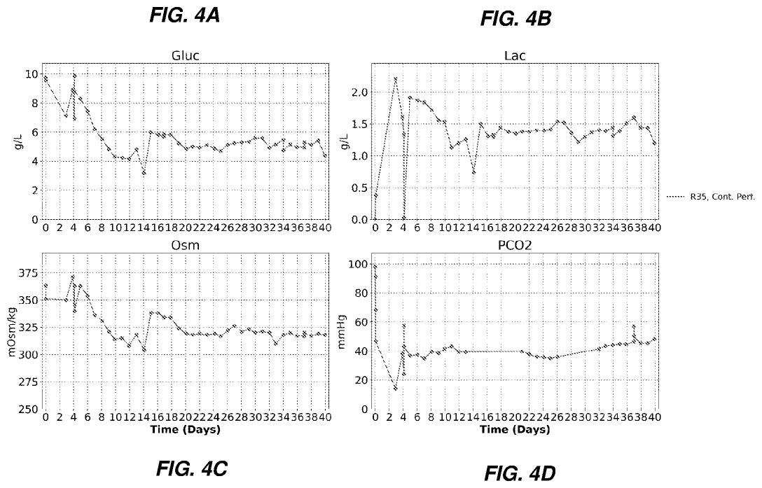

FIGS. 5A-5D show measurement of additional primary metabolic profile parameters glutamine (Gln) (FIG. 5A), glutamate (Glu) (FIG. 5B), pH (FIG. 5C), Ammonium (NH₄) (FIG. 5D) levels during continuous perfusion cell culture.

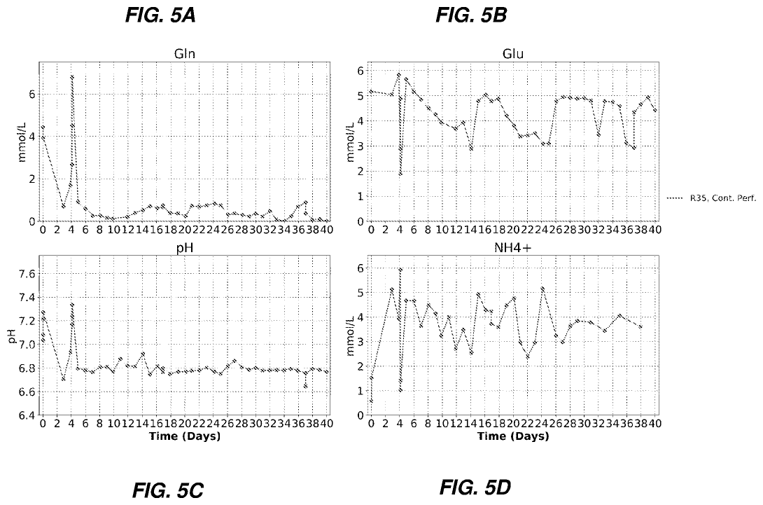

### DETAILED DESCRIPTION OF THE INVENTION

The present disclosure provides various processes for producing a biomolecule in a continuous process using dual vessel tangential flow filtration.

Several dual vessel systems have been described in, for example, U.S. Pat. No. 8,679,778 and US Patent Application No. 20190322975, however such systems are limited in filtration methods or lack the ability to control biomolecule product conditions in a second bioaccumulation vessel or include limitations such as a recycle stream from the bioaccumulation vessel to the cell culture vessel.

In various embodiments, the disclosure provides a process for producing a biomolecule comprising the following steps: (a) culturing cells in a cell culture vessel, where the cell culture vessel is continuously perfused with enriched cell culture medium, and where the cells are cultured under conditions that allow the production of biomolecules. The process produces a cell culture composition comprising the cells, medium and biomolecules. Step (b) comprises passing the cell culture composition of (a) through a first filter, where the first filter retains the cells but allows the passage of biomolecules and medium into a biomolecule accumulation vessel, thereby producing a biomolecule composition. Step (c) comprises concentrating the biomolecules by passing the biomolecule composition of (b) through a second filter, where the second filter retains and returns the biomolecules to the biomolecule accumulation vessel but allows removal of spent medium into a waste collection vessel. Finally, step (d) comprises harvesting the biomolecules from the biomolecule accumulation vessel while continuing steps (a)-(c); thereby purifying the biomolecule.

### Definitions

As used herein, “producing one or more biomolecules” refers to the steps of manufacture of a biomolecule of interest by expressing the biomolecule in a collection of cells or a cell line and purifying the biomolecule of interest.

As used herein, “culturing cells” refers to the process by which cells are grown under controlled conditions in a cell culture vessel to allow for the production of a biomolecule of interest.

As used herein, “conditions that allow for the production” refers to cell culture conditions (e.g. pH, temperature, dO₂, CO₂, time, etc.)

The present disclosure contemplates that various vessels can be used according to the methods described herein. As used herein, “cell culture vessel” refers to any container or device or system that supports a biologically active environment. For example, a cell culture vessel may include a constant stir tank reactor (CSTR). In various embodiments, the biomolecule accumulation vessel is a constant stir tank reactor (CSTR) or a harvest bag.

As used herein, the cells and/or one or more biomolecules produced from the cells are kept stable throughout the process for a desired period of time. As used herein, the term “stable” or “stably” as it relates to the cells in a vessel means maintaining a population of cells over a desired period of time, and further refers to maintaining a population of cells which maintain stable expression of the biomolecule of interest as it relates to biomolecules. As it relates to a biomolecule, the term “stable” or “stably” refers to maintaining the integrity of the biomolecule in, for example, a biologically active conformation (i.e., without degradation, unfolding, and the like). As used herein, “biomolecule accumulation vessel” refers to any container or device or system that supports a biomolecule composition.

As used herein, “waste collection vessel” refers to any device or system that supports collection of waste byproducts from cell culture (e.g. cell debris).

As used herein, “enriched cell culture medium or media” refers to medium which has been supplemented to contain the nutrients to support the growth of the cells being cultured. In related aspects, the enriched cell culture medium is fresh medium provided to maintain the continuous cell culture that allow for the production of one or more biomolecules of interest.

As used herein, “continuously perfused with enriched cell culture” refers to the process of continuous perfusion of the cell culture with enriched cell culture medium while the waste products/impurities in the medium are continuously removed from the culture and thereby the displaced medium is replenished.

### Biomolecule of Interest

In various aspects, the process described herein produces and purifies one or more biomolecules of interest. In various aspects, the process described herein produces and purifies one biomolecule of interest. In various aspects, the process described herein produces and purifies two, three or four biomolecules of interest.

In a suitable example the biomolecule of interest has a MW of at least 2 kD, or at least 5 kD, or at least 15 kD, or at least 150 kD or at least 500 kD. In various embodiments the biomolecule is a polypeptide of interest. In various aspects, the biomolecule of interest includes, but is not limited to, a polypeptide, polysaccharide, polypeptide/polysaccharide hybrid, Fc fusion polypeptide, polynucleotide, cytokine, growth factor, hormone, enzyme, vaccine, anticoagulation factor or small molecule. In various aspects, polypeptides include, but are not limited to, an antibody or antigen-binding fragment thereof, a derivative of an antibody or antibody fragment, or a fusion polypeptide. In various aspects, the biomolecule is an intact viral particle or a fragment thereof such as a viral capsid protein, viral glycoprotein such as hemagglutinin or neuraminidase, viral spike protein, or other inactive viral fragments used in vaccine or gene therapy or cell therapy applications. In exemplary aspects, the biomolecule of interest is an antibody. As used herein, the term “antibody” refers to a protein having a conventional immunoglobulin format, comprising heavy and light chains, and comprising variable and constant regions. For example, an antibody can be an IgG which is a “Y-shaped” structure of two identical pairs of polypeptide chains, each pair having one “light” (typically having a molecular weight of about 25 kD) and one “heavy” chain (typically having a molecular weight of about 50-70 kD). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different anti-gens. The constant region allows the antibody to recruit cells and molecules of the immune sys-tem. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4th ed. Elsevier Science Ltd./Garland Publishing, (1999)).

Antibodies can comprise any constant region known in the art. Human light chains are classified as kappa and lambda light chains. Heavy chains are classified as mu, delta, gamma, alpha, or epsi-lon, and define the antibody's isotype as IgM, IgD, IgG, IgA, and IgE, respectively. IgG has several subclasses, including, but not limited to IgG1, IgG2, IgG3, and IgG4. IgM has sub-classes, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

The antibody can be a monoclonal antibody or a polyclonal antibody. In some embodiments, the antibody comprises a sequence that is substantially similar to a naturally-occurring antibody produced by a mammal, e.g., mouse, rabbit, goat, horse, hamster, human, and the like. In this regard, the antibody can be considered as a mammalian antibody, e.g., a mouse antibody, rabbit antibody, goat antibody, horse antibody, hamster antibody, human anti-body, and the like. In certain aspects, the antibody is a human antibody. In certain aspects, the antibody is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

In some aspects, the antibody is infliximab, infliximab, bevacizumab, cetuximab, ranibizumab, palivizumab, abagovomab, abciximab, actoxumab, adalimumab, afelimomab, afutuzumab, alacizumab, alacizumab pegol, ald518, alemtuzumab, alirocumab, altumomab, amatuximab, anatumomab mafenatox, anrukinzumab, apolizumab, arcitumomab, aselizumab, altinumab, atlizumab, atorolimiumab, aritox, tocilizumab, bapineuzumab, basiliximab, bavituximab, bectumomab, belimumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, crenezumab, cr6261, dacetuzumab, daclizumab, dalotuzumab, daratumumab, demcizumab, denosumab, detumomab, dorlimomab aritox, drozitumab, duligotumab, dupilumab, ecromeximab, eculizumab, edobacomab, edrecolomab, efalizumab, efungumab, elotuzumab, elsilimomab, enavatuzumab, enlimomab pegol, enokizumab, enoticumab, ensituximab, epitumomab cituxetan, epratuzumab, erenumab, erlizumab, ertumaxomab, etaracizumab, etrolizumab, evolocumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumomab tiuxetan, icrucumab, igovomab, imciromab, imgatuzumab, inclacumab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, tefibazumab, teneliximab, teplizumab, teprotumumab, tezepelumab, TGN1412, tremelimumab, ticilimumab, tildrakizumab, tigatuzumab, TNX-650, tocilizumab, toralizumab, tositumomab, tralokinumab, trastuzumab, TRBS07, tregalizumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, zolimomab, or biologically active fragments, analogs or variants thereof.

### Cell Culture Vessel

In various aspects, a cell culture vessel is a bioreactor in which is carried out a chemical process which involves organisms or biochemically active substances derived from such organisms. This process can either be aerobic or anaerobic. Cell culture vessels are commonly cylindrical, ranging in size from some liters to cubic meters, and are often made of stainless steel but could also be made of other materials such as disposable materials.

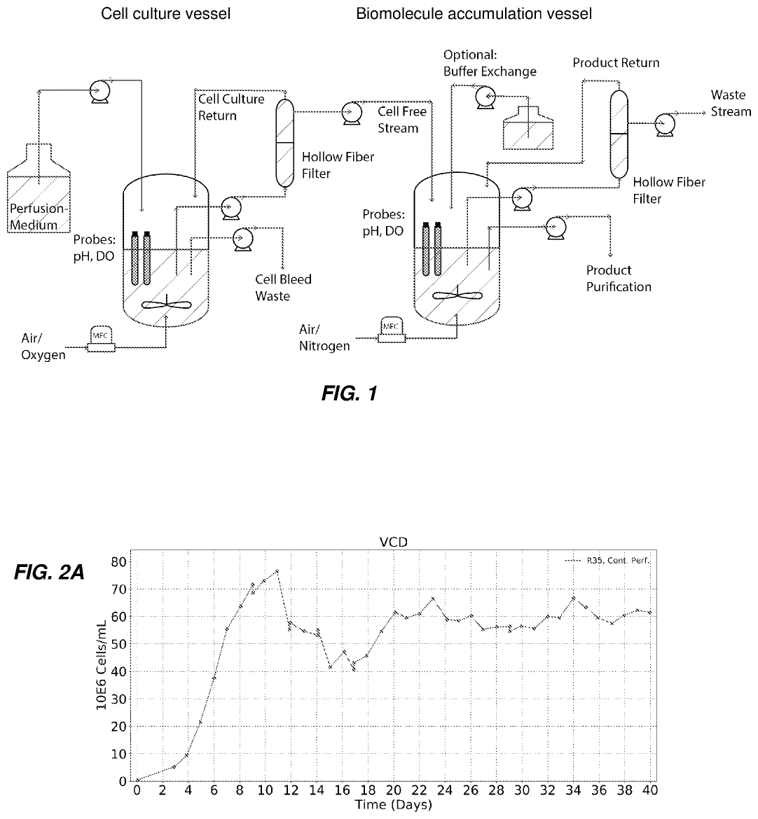

A cell culture vessel may also refer to a device or system meant to grow cells or tissues in the context of cell culture. On the basis of mode of operation, a cell culture vessel may be classified as batch, fed-batch or continuous (e.g. continuous stirred-tank reactor model). In various aspects, the cell culture vessel is a constant stir tank reactor (CSTR). An example of a cell culture vessel is the chemostat. The cell culture vessel may be equipped with one or more inlets for supplying new fresh or concentrated medium to the cells and one or more outlets for passing the biomolecule of interest to a biomolecule accumulation vessel or emptying the cell culture vessel. Additionally, the cell culture vessel may be equipped with at one or more outlets constructed in such a way that filter units can be attached to the cell culture vessel. In various aspects, the cell culture vessel is continuously perfused with enriched or concentrated cell culture medium. In various aspects, cells are cultured under conditions that allow for the production of one or more biomolecules of interest, thereby producing a cell culture composition comprising said cells, medium and biomolecules of interest. In various aspects, the cell culture composition is passed through a filter which retains the cells but allows the passage of biomolecules of interest into a biomolecule accumulation vessel. An exemplary cell culture vessel is shown in FIG. 1.

In various aspects, the cell culture vessel has a volume of at least 50 L, more preferably at least 100 L, even more preferably at least 250 L, even more preferable at least 500 L, and most preferably at least 2000 L.

In various aspects, the cell culture in the cell culture vessel is stable for 20-120 days. In various aspects, the cell culture is stable for 20, 25, 30, 35, 40, 45, 50, 55, 60, 65, 70, 75, 80, 85, 90, 95, 100, 105, 110, 115, or 120 days. In related aspects, the cell culture is maintained for 30-60 days. In preferred embodiments, the cell culture is maintained for 30 days.

### Biomolecule Accumulation Vessel

In various aspects, a biomolecule accumulation vessel is a bioreactor in which is carried out a chemical process which involves biomolecules or biochemically active substances derived from organisms. In various aspects, biomolecules are concentrated in the biomolecule accumulation vessel. The biomolecule accumulation vessel may be equipped with one or more inlets for supplying a cell free stream and one or more outlets for harvesting product or emptying the biomolecule accumulation vessel. Additionally, the biomolecule accumulation vessel may be equipped with one or more outlets constructed in such a way that filter units can be attached to the biomolecule accumulation vessel. In various aspects, the biomolecule accumulation vessel comprises a filter which retains and returns the biomolecules to the biomolecule accumulation vessel but allows removal of spent medium into a waste collection vessel. In various aspects, the biomolecule accumulation vessel is a constant stir tank reactor (CSTR). In alternative aspects, the biomolecule accumulation vessel is a single-use sterile harvest bag. In optional embodiments, the biomolecule accumulation vessel comprises a buffer exchange. An exemplary biomolecule accumulation vessel is shown in FIG. 1. In various aspects, the biomolecule of interest is concentrated in the biomolecule accumulation vessel. In various aspects, the biomolecule accumulation vessel optionally comprises a buffer exchange claim.

In various aspects, the pH, temperature, agitation, constant air overlay, dO₂, or CO₂ conditions in the biomolecule accumulation vessel for bench scale are varied between pH 4-10, 4-40° C., 10-500 RPM, 10-100,000 sccm, 0-100% dO₂, and/or 0-100% CO₂. In various aspects, the pH in the biomolecule accumulation vessel is at least 4, 4.5, 5, 5.5, 6, 6.5, 7, 7.5, 8, 8.5, 9, 9.5 or 10. In preferred embodiments, the optimal pH is 6.4-6.8. In various aspects, the temperature in the biomolecule accumulation vessel is at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C. In preferred embodiments, the temperature is 14° C. In various aspects, the RPM in the biomolecule accumulation vessel is at least 10, 20, 30, 40, 50, 100, 150, 200, 250, 300, 350, 400, 450 or 500 RPM. In preferred embodiments, the RPM is 50. In various aspects, the air overlay in the biomolecule accumulation vessel is at least 10, 100, 1000, 10,000, 50,000 or 100,000 sccm. In preferred embodiments, the sccm is 100. In various aspects, the dO₂ in the biomolecule accumulation vessel is at least 0, 10, 20, 30, 40, 50, 60, 70, 80, 90 or 100%. In preferred embodiments, the dO₂ is 70%. In various aspects, the CO₂ in the biomolecule accumulation vessel is at least 0, 10, 20, 30, 40, 50, 60, 70, 80, 90 or 100% CO₂. In preferred embodiments, the CO₂ is 0%.

In various aspects, the pH, temperature, agitation, constant air overlay, dO₂, or CO₂ conditions in the biomolecule accumulation vessel for production scale are varied between pH 4-10, 4-40° C., 10-500 RPM, 10-100,000 sccm, 0-100% dO₂, and/or 0-100% CO₂. In various aspects, the pH in the biomolecule accumulation vessel is at least 4, 4.5, 5, 5.5, 6, 6.5, 7, 7.5, 8, 8.5, 9, 9.5 or 10. In preferred embodiments, the optimal pH is 6.4-6.8. In various aspects, the temperature in the biomolecule accumulation vessel is at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C. In preferred embodiments, the temperature is 14° C. In various aspects, the RPM in the biomolecule accumulation vessel is at least 10, 20, 30, 40, 50, 100, 150, 200, 250, 300, 350, 400, 450 or 500 RPM. In preferred embodiments, the RPM is 20. In various aspects, the air overlay in the biomolecule accumulation vessel is at least 10, 100, 1000, 10,000, 50,000 or 100,000 sccm. In preferred embodiments, the sccm is 5000. In various aspects, the dO₂ in the biomolecule accumulation vessel is at least 0, 10, 20, 30, 40, 50, 60, 70, 80, 90 or 100%. In preferred embodiments, the dO₂ is 70%. In various aspects, the CO₂ in the biomolecule accumulation vessel is at least 0, 10, 20, 30, 40, 50, 60, 70, 80, 90 or 100% CO₂. In preferred embodiments, the CO₂ is 0%.

### Filters

Several specialized filters and filtration methods have been developed to separate materials according to their chemical and physical properties. Hollow fiber membranes have been successfully employed in a wide variety of industries including food, juice, pharmaceutical, metalworking, dairy, wine and most recently municipal drinking water. Depending on the application, hollow fiber membranes can be highly practical and cost effective alternatives to conventional chemical and physical separation processes. Hollow fiber membranes offer the unique benefits of high membrane packing densities, sanitary designs and, due to their structural integrity and construction, can withstand permeate back-pressure thus allowing flexibility in system design and operation. Hollow fiber cartridges can operate from the inside to the outside during filtration. This means that process fluid (retentate) flows through the center of the hollow fiber and permeate passes through the fiber wall to the outside of the membrane fiber. Tangential flow can help limit membrane fouling. Other operating techniques that can be employed with hollow fiber membrane systems include back flushing with permeate and retentate reverse flow. Examples of filtration systems applicable for use in the processes described herein include alternating tangential flow (ATF) and tangential flow filtration (TFF) filters, including, for example, tangential flow depth filtration (TFDF™) filters.

In various aspects, a first filter is operably attached to the cell culture vessel. In various aspects, the pore size of the first filter is between 0.2-1 μm. In related aspects, the pore size of the first filter is 0.2, 0.3, 0.4, 0.5, 0.6, 0.7, 0.8, 0.9 or 1.0 μm. In certain aspects, the pore size of the first filter is 0.5 μm.

In various aspects, a second filter is operably attached to the biomolecule accumulation vessel. In various aspects, the pore size of the second filter is between 10-200 kD. In related aspects, the pore size of the second filter is 10, 20, 30, 40, 50, 60, 70, 80, 90, 100, 110, 120, 130, 140, 150, 160, 170, 180, 190, or 200 kD. In certain aspects, the pore size of the second filter is 50 kD.

In various aspects, the first filter is an Alternating Tangential Flow (ATF) filter. In various aspects, the second filter is an Alternating Tangential Flow (ATF) filter. In alternative aspects, the first filter is a Tangential Flow Filtration (TFF) filter. In alternative aspects, the second filter is a Tangential Flow Filtration (TFF) filter. In various aspects, the first filter and the second filter are each Alternating Tangential Flow (ATF) filters. In various aspects, the first filter and the second filter are each Tangential Flow Filtration (TFF) filters.

The first and second filters may be located in an external filter module attached to cell culture and/or biomolecule accumulation vessel. Alternatively both the first filter and the second filter may be located inside the cell culture and/or biomolecule accumulation vessel. The filter units can also contain pumps or systems for preventing fouling of the filter such as the ATF system.

In various aspects, the first and second filter tangential flow rate is 0.5-1000 L/min. In various aspects, the first and second filter tangential flow rate is at least 0.5, at least 10.0, at least 20.0, at least 30.0, at least 40.0, at least 50.0, at least 60.0 L/min. In related aspects the tangential flow rate of the first filter is the same or different from tangential flow rate of the second filter. In preferred embodiments for bench scale, the tangential flow rate is 0.8 L/min. In preferred embodiments for production scale, the tangential flow rate is 60 L/min.

### Cells

Any eukaryotic and prokaryotic cells are contemplated for use in the instant processes, including mammalian, bacterial, yeast, fungal, insect, plant cells.

Examples of such cells include, but are not limited to, mammalian cells, such as Chinese hamster ovary cells (CHO) (ATCC No. CCL61); CHO DHFR-cells; serum-free, suspension-adapted CHO DHFR cell line was created at CMC ICOS (SFSA DG44 cells); human embryonic kidney (HEK) 293 or 293T cells (ATCC No. CRL1573); or 3T3 cells (ATCC No. CCL92). Other suitable mammalian cell lines, are the monkey COS-1 (ATCC No. CRL1650) and COS-7 (ATCC No. CRL1651) cell lines, and the CV-1 cell line (ATCC No. CCL70). Still other suitable mammalian cell lines include, but are not limited to, Sp2/0, NS1 and NS0 mouse hybridoma cells, mouse neuroblastoma N2A cells, HeLa, mouse L-929 cells, 3T3 lines derived from Swiss, Balb-c or NIH mice, BHK or HaK hamster cell lines, which are also available from the ATCC.

Further exemplary mammalian host cells for producing a biomolecule include primate cell lines and rodent cell lines, including transformed cell lines. Normal diploid cells, cell strains derived from in vitro culture of primary tissue, as well as primary explants, are also suitable.

Similarly useful cells include, for example, the various strains of E. coli(e.g., HB101, (ATCC No. 33694) DH5α, DH10, and MC1061 (ATCC No. 53338)), various strains of B. subtilis, Pseudomonas spp., Streptomyces spp., Salmonella typhimurium and the like.

Additionally, where desired, insect cell systems may be utilized in the methods of the present invention. Such systems include for example and without limitation, Sf-9 and Hi5 (Invitrogen, Carlsbad, CA).

Exemplary fungal cells include, without limitation, Thermoascus aurantiacus, Aspergillus(filamentous fungus), including without limitation Aspergillus oryzaem, Aspergillus nidulans, Aspergillus terreus, and Aspergillus niger, Fusarium (filamentous fungus), including without limitation Fusarium venenatum, Penicillium chrysogenum, Penicillium citrinum, Acremonium chrysogenum, Trichoderma reesei, Mortierella alpina, and Chrysosporium lucknowense.

Exemplary protozoan cells include without limitation Tetrahymena strains and Trypanosoma strains.

### Fermenting Cells in Cell Culture Vessel in a Suitable Medium Under Suitable Conditions

The skilled person knows what could be suitable medium and suitable conditions with respect to specific expression cells and polypeptide of interest.

The term “medium” generally refers to, a cell culture medium, which may comprises salts, amino acids, vitamins, lipids, detergents, buffers, growth factors, hormones, cytokines, trace elements and carbohydrates. Examples of salts include magnesium salts, for example MgCl₂×6H₂O and iron salts, for example FeSO₄×7H₂O, potassium salts, for example KH₂PO₄, KCl; sodium salts, for example NaH₂PO₄ or Na₂HPO₄ and calcium salts, for example CaCl₂×·2H₂O. Examples of amino acids are all 20 known proteinogenic amino acids, for example histidine, glutamine, threonine, serine, methionine. Examples of vitamins include: ascorbate, biotin, choline, myo-inositol, and D-panthothenate, riboflavin. Examples of lipids include: fatty acids, for example linoleic acid and oleic acid; soy peptone and ethanol amine. Examples of detergents include Tween 80 and Pluronic F68. An example of a buffer is HEPES. Examples of growth factors/hormones/cytokines include IGF, hydrocortisone and (recombinant) insulin. Examples of trace elements are known to the person skilled in the art and include Zn, Mg and Se. Examples of carbohydrates include glucose, fructose, galactose and pyruvate.

The pH, temperature, agitation, constant air overlay, dissolved oxygen concentration (dO₂), CO₂ and/or osmolarity of the cell culture medium in the cell culture vessel are dependent on the type of cell chosen. Preferably, the pH, temperature, dissolved oxygen concentration and osmolarity are chosen such that it is optimal for the growth and productivity of the cells in the cell culture vessel. The person skilled in the art knows how to find the optimal pH, temperature, dissolved oxygen concentration and osmolarity for the perfusion culturing. Usually, the optimal pH is between 6.6 and 7.6, the optimal temperature between 30° C. and 39° C., the optimal osmolarity between 260 and 400 mOsm/kg. In preferred embodiments, the optimal pH is 7.0+/−0.2. In preferred embodiments, the optimal temperature is 34° C. In preferred embodiments, the optimal dO₂ is 40%. In preferred embodiments, the optimal CO₂ is <20%. Alternatively, silicon-based antifoams and defoamers or nonionic surfactants such as coblock polymers of ethylene oxide/propylene oxide monomers may be added to the medium during fermentation. The medium may be water.

The skilled person knows numerous suitable expression cells. In a preferred embodiment, the cell expressing the biomolecule (e.g. polypeptide) of interest is at least one cell selected from the group consisting of E. coli, Bacillus, yeast from the genus of Saccharomyces, Pichia, Aspergillus, Fusarium, Kluyveromyces, CHO (Chinese Hamster Ovary) cell, hybridomas, BHK (Baby Hamster Kidney) cell, myeloma cell, HEK-293 cell, human lymphoblastoid cell and a mouse cell, for example a NSO cell.

### During the Cell Culture-Removing Impurities

The term “impurities” shall be understood as the skilled person would understand it in the present context. Impurities are understood as chemical or biological compounds produced by the cells present in the bioreactor, which limit the growth of the cells. Impurities can also arise from cells that die or break open during the fermentation process. Impurities could comprise ethyl alcohol, butyl alcohol, lactic acid, acetone ethanol, gaseous compounds, peptides, lipids, ammonia, aromatic compounds and DNA and RNA fragments.

The following Examples serves only to illustrate the invention and is not intended to limit the scope of the invention in any way.

Harvesting the Biomolecule

According to step (d) of first aspect, the biomolecule (e.g. polypeptide) of interest is isolated from the harvested medium. In various aspects, the harvesting step (d) occurs continuously while performing steps (a), (b) and (c) (i.e. culturing, filtering and concentrating biomolecules). In various aspects, the harvesting step (d) occurs every 1-60 days. In related aspects, the harvesting step (d) occurs every 1, 2, 3, 4, 5, 6, 7, 8, 9 10, 11, 12, 13, 14, 15, 20, 25, 30, 35, 40, 45, 50, 55 or 60 days. In preferred embodiments, the harvesting step (d) occurs every 40 days. As the cell culture vessel can be maintained stable and productive for a duration of months, the biomolecule accumulation vessel can undergo multiple harvests in a single production cycle.

The isolated biomolecule (e.g. polypeptide) of interest is normally formulated into a final commercial relevant composition of interest (e.g. a pharmaceutical composition of interest). Further it is normally packaged in a suitable container.

### Cell Density

Cells that are advantageously subjected to the process of the invention may be any cell type benefiting from this process, i.e. culturing to a high viable cell density.

According to the process of the invention, a high viable cell density is preferably a density of at least 15 mill cells/ml, preferably at least 20 mill cells/ml, more preferably at least 25 mill cells/ml, even more preferably at least 30 mill cells/ml, even more preferably at least 60 mill cells/ml and most preferably at least 80 mill cells/ml.

### EXAMPLES

### General Methods

Equipment

A 3 L bench scale CSTR with 2 L working volume (Applikon), Sartorius DCU2 controllers, Repligen ATF2 controller and single use hollow fiber filter, Beckman Coulter Vi-Cell cell counter, Nova Biomedical FLEX2 metabolite analyzer, Chinese Hamster Ovary (CHO) DG44 cell line, enriched perfusion medium.

### Example 1: A Dual Vessel Tangential Flow Filtration Process for Producing a Biomolecule

A fully continuous process for producing a biomolecule comprising two constant stir tank reactors (CSTR) connected in series was setup as shown in FIG. 1. Material flows from the cell culture vessel to the biomolecule accumulation vessel. Each vessel is connected to a hollow fiber tangential flow filtration device with a pore size of 0.2 μm and 50 kD for the cell culture vessel and the biomolecule accumulation vessel respectively. Parameters such as temperature, agitation, pH, and dissolved oxygen are controlled and varied in order to promote healthy cell growth in the cell culture vessel, and to stabilize the product in the accumulation vessel.

The 0.2 μm hollow fiber tangential-flow filter retains live cells inside the cell culture vessel while allowing the product to pass in a cell-free stream into the biomolecule accumulation tank. Fresh perfusion medium is delivered into the cell culture vessel to maintain high cell-density environment and a constant vessel volume. In addition, cell biomass is removed via a cell bleed stream in order to maintain stable cell density for extended duration. In order to maintain a high cell density culture for extended duration, it is critical to: deliver enriched perfusion medium, provide sufficient oxygen mass transfer, and implement an appropriate strategy for biomass removal rate.

The biomolecule (product) is concentrated inside the biomolecule accumulation vessel via a 50 kD hollow fiber tangential flow filter system. Molecules smaller than 50 kD cutoff are passed through the filter and into a waste stream. The biomolecule is accumulated until a favorable concentration for subsequent purification steps. The biomolecule accumulation vessel conditions can be controlled in order to provide a stable product environment that would otherwise be impossible to implement in a cell-culture tank. For instance, biomolecules such as antibody drug substances are stabilized in low temperature and low pH that would be detrimental to live cell culture. Since the cell culture vessel can be maintained stable and productive for a duration of weeks or months, the biomolecule accumulation vessel can undergo multiple harvests in a single production cycle.

### Example 2: Production of a Biomolecule in Mammalian Cells Using a Dual Vessel Tangential Flow Filtration Process

1st Vessel: Maintain Stable High Cell Density Steady-State

### Inoculation

A frozen cell culture vial containing CHO DG44 cells was thawed in warm water bath (37° C.), and transferred into shake-flask containing pre-warmed medium. The flask working volume was 25 mL and the target cell density was 0.5E6 cell/mL. The flask was incubated at 37° C., 5% CO₂, and 120 RPM (on a 25 mm rotor). Expansion was carried out every 2 or 3 days with a target seed density of 0.5E6 cells/mL, and the number of passages and final working volume was dependent on the number of bioreactor vessels in the process design. A 2 L flask culture working volume with cell density of 2E6 cells/mL was used to inoculate 4 2 L vessels with seeding density of 0.5E6 cells/mL. A typical expansion from 30 mL to 2 L was completed in 10 days or 4 passages of the cells.

Prior to inoculation, the bioreactor vessel and all tubing, sample ports, and disposable hollow fiber filter was autoclave sterilized per standard procedure. The vessel was then batched with sterile medium 24 hours prior to inoculation. The vessel medium was brought up to process temperature and pH of 37° C. and 7.0+/−0.2 respectively, and was left agitating for 24 hours for sterility confirmation. Control of pH levels were implemented via carbon dioxide (CO₂) for acid side and Sodium carbonate (Na₂CO₃) for the base side of pH control. Control of dissolved oxygen levels was also initiated during this time. Medium working volume target was 1.5 L, and final working volume after the vessel was inoculated with cell culture was 2 L. The target seed density was 0.5E6 cells/mL.

### Standard Batch Process for First 3 Days.

Post inoculation, the vessels were maintained under standard batch conditions for the first 3 days (Day 0-Day 3) while the cells were in growth phase and at low cell density. Controller settings remain unchanged at this time with constant control setpoints: dissolved oxygen, temperature, agitation, pH levels.

### Perfusion

Perfusion via hollow fiber alternating tangential flow filtration was initiated on day 3 to promote exponential cell growth. The filter connected to the cell culture vessel pore size was 0.2-0.5 μm in diameter. The alternating tangential flow was set to 0.8 L/min via Repligen ATF2 controller system. At the same time, a medium pump was activated for a continuous medium flux through the 0.2 μm filter and into the second vessel (product accumulation vessel). The vessel controller was activated to pump enriched perfusion medium and maintain constant vessel volume.

### Bleed Rate

In order to maintain a continuously sustained cell density (e.g. 30+ days), a cell waste stream was implemented in the culture vessel and some of the biomass was removed. This stream was known as the cell bleed stream and was used to maintain cell viability for prolonged process durations. Biomass removal rate can be calculated from maximum cell growth rate and was dependent on process conditions such as temperature and medium composition. In order to maximize productivity, the highest possible cell density was targeted for continuous cell culture.

### Maintain Stable Cell Culture

It was critical to maintain high mass transfer coefficient in order to sustain high cell density. The gassing strategy was to shift from drilled-hole oxygen delivery to sintered sparge while biomass was still accumulating. In addition, controller settings required highly optimized feedback loop and Proportional-Integral-Derivative (PID) control in order to maintain control over dissolved oxygen in the cell culture vessel.

2nd Vessel: Maintain Favorable Conditions for Extended Product Storage and Concentration

### Vessel Conditions: Product Specific

The product accumulation vessel was coupled to the cell culture vessel, however, the cells were filtered out and remained in the cell culture vessel. Therefore, the conditions set in the biomolecule accumulation tank, were not restricted to conditions that are favorable for cell growth. Process settings such as temperature, pH, agitation, dissolved oxygen were tailored specifically for the biomolecule product being manufactured. The material was further stabilized via additional of an optional buffer solution for a favorable environment on the chemical level. The concentrated product underwent multiple harvests in the duration of the extended continuous process and was dependent on factors such as optimum harvest titer for subsequent purification steps. Surprisingly, cells were continuously cultured and maintained in the cell culture vessel and antibodies were stably maintained in the biomolecule accumulation vessel for a period of over 30 days. Under these conditions at least at 2 fold, at least at 3 fold, at least at 4 fold, at least at 5 fold, at least at 6 fold, at least at 7 fold, at least at 8 fold, at least at 9 fold, at least at 10 fold increase in titer was obtained relative to fed-batch process (e.g. 20 g/L versus 2 g/L titer).

### Tangential Flow Filtration

Tangential flow was 0.8 L/min for a working vessel volume of 2 L. A 50 kD pore size filter was used in order to retain the antibody product. The vessel volume remained constant via a control loop that activated an outlet pump when vessel volume exceeds the preset threshold.

1. A process for producing one or more biomolecules of interest comprising:
(a) culturing cells in a cell culture vessel, wherein said cell culture vessel is continuously perfused with enriched cell culture medium, and wherein said cells are cultured under conditions that allow for the production of one or more biomolecules of interest, thereby producing a cell culture composition comprising said cells, medium and biomolecules of interest;
(b) passing the cell culture composition of (a) through a first filter, wherein said first filter retains the cells but allows the passage of biomolecules and medium into a biomolecule accumulation vessel, thereby producing a biomolecule composition;
(c) concentrating said biomolecules by passing the biomolecule composition of (b) through a second filter, wherein said second filter retains and returns the biomolecules to the biomolecule accumulation vessel but allows removal of spent medium into a waste collection vessel; and
(d) harvesting said biomolecules from the biomolecule accumulation vessel while continuing steps (a)-(c); thereby purifying said one or more biomolecules of interest.

2. The process of claim 1, wherein the cells are eukaryotic or prokaryotic cells.

3. The process of claim 1, wherein the first filter is operably attached to the cell culture vessel.

4. The process of claim 3, wherein the second filter is operably attached to the biomolecule accumulation vessel.

5. The process of claim 4, wherein the pore size of the first filter is 0.2-1 μm.

6. (canceled)

7. The process of claim 4, wherein the pore size of the second filter is 10-200 kD.

8. (canceled)

9. The process of claim 4, wherein the first filter is an Alternating Tangential Flow (ATF) filter or a Tangential Flow Filtration (TFF) filter.

10. The process of claim 4, wherein the second filter is an Alternating Tangential Flow (ATF) filter or a Tangential Flow Filtration (TFF) filter.

11. (canceled)

12. (canceled)

13. (canceled)

14. (canceled)

15. The process of claim 4, wherein the first and second filter tangential flow is 0.5-1000 L/min.

16. (canceled)

17. (canceled)

18. (canceled)

19. The process of claim 4, wherein the pH, temperature, agitation, constant air overlay, dO₂, or CO₂ conditions in the biomolecule accumulation vessel are varied between pH 4-10, 10-40° C., 10-400 RPM, 10-100,000 sccm, 0-100% dO₂, and/or 0-100% CO₂.

20. (canceled)

21. The process of claim 4, wherein the harvesting step (d) occurs continuously while performing steps (a), (b) and (c).

22. The process of claim 4, wherein the harvesting step (d) occurs every 1-60 days.

23. The process of claim 4, wherein the cell culture vessel is a constant stir tank reactor (CSTR).

24. The process of claim 4, wherein the biomolecule accumulation vessel is a constant stir tank reactor (CSTR) or a harvest bag.

25. (canceled)

26. (canceled)

27. The process of claim 4, wherein the biomolecule is a polypeptide, polysaccharide, polypeptide/polysaccharide hybrid, Fc fusion polypeptide, polynucleotide, cytokine, growth factor, hormone, enzyme, vaccine, anticoagulation factor or small molecule.

28. The process of claim 27, wherein the polypeptide is an antibody, antibody fragment, antibody fusion peptide or antigen-binding fragments thereof.

29. A process for producing a biomolecule comprising:
(a) culturing cells in a cell culture vessel, wherein said cell culture vessel is continuously perfused with enriched cell culture medium, and wherein said cells are cultured for 30-60 days under conditions that allow the production of a biomolecule, thereby producing a cell culture composition comprising said cells, medium and biomolecules;
(b) passing the cell culture composition of (a) through a first 0.2 μm hollow fiber filter wherein said first hollow fiber filter retains the cells but allows the passage of biomolecules and medium into a biomolecule accumulation vessel, thereby producing a biomolecule composition;
(c) concentrating said biomolecules by passing the biomolecule composition of (b) through a second 50 kD hollow fiber filter, wherein said second hollow fiber filter retains and returns the biomolecules to the biomolecule accumulation vessel but allows removal of spent medium into a waste collection vessel and wherein the pH, temperature, agitation, constant air overlay, dO₂, or CO₂ conditions in the biomolecule accumulation vessel are varied between pH 4-10, 30-40° C., 10-400 RPM, 10-100,000 sccm, 0-100% dO₂, and/or 0-100% CO₂; and
(d) harvesting said biomolecules from the biomolecule accumulation vessel while continuing steps (a)-(c); thereby purifying said biomolecule.

30. A process for producing a biomolecule comprising:
(a) culturing cells in a cell culture vessel, wherein said cell culture vessel is continuously perfused with enriched cell culture medium, and wherein said cells are cultured for up to 30 days under conditions that allow the production of a biomolecule, thereby producing a cell culture composition comprising said cells, medium and biomolecules;
(b) passing the cell culture composition of (a) through a first 0.2 μm hollow fiber filter wherein said first hollow fiber filter retains the cells but allows the passage of biomolecules and medium into a biomolecule accumulation vessel, thereby producing a biomolecule composition;
(c) concentrating said biomolecules by passing the biomolecule composition of (b) through a second 50 kD hollow fiber filter, wherein said second hollow fiber filter retains and returns the biomolecules to the biomolecule accumulation vessel but allows removal of spent medium into a waste collection vessel and wherein the pH, temperature, agitation, constant air overlay, dO₂, or CO₂ conditions in the biomolecule accumulation vessel are varied between pH 6.4-6.8, 14° C., 20-50 RPM, 100-5000 sccm, 70% dO₂, and/or 0% CO₂; and
(d) harvesting said biomolecules from the biomolecule accumulation vessel every 40 days while continuing steps (a)-(c); thereby purifying said biomolecule.
